(19) United States
(12) Patent Application Publication
(10) Pub. No.: US 2020/0230084 A1
(43) Pub. Date: Jul. 23, 2020

(54) METHODS OF TREATING DISEASE WITH DICHLORPHENAMIDE

(71) Applicant: Strongbridge Dublin Limited, Trevose, PA (US)

(72) Inventor: Fredric Jay COHEN, Washington Crossing, PA (US)

(21) Appl. No.: 16/253,505

(22) Filed: Jan. 22, 2019

(51) Int. Cl. A61K 31/18; A61B 5/16

(52) U.S. Cl. CPC A61K 31/18; A61B 5/16

(57) ABSTRACT

Provided herein is a method for treating primary hyperkalemic periodic paralysis, primary hypokalemic periodic paralysis, and related variants. The method comprises administering to the subject a therapeutically effective amount of dichlorphenamide, or a pharmaceutically acceptable salt thereof, informing the subject to notify a medical professional if the subject experiences symptoms of cognitive impairment, and adjusting the therapeutically effective amount of dichlorphenamide, or a pharmaceutically acceptable salt thereof, when the subject is experiencing symptoms of cognitive impairment. The subject or a medical care worker may be informed that administration of the dichlorphenamide, or a pharmaceutically acceptable salt thereof, may result in increased risk of the subject experiencing symptoms of cognitive impairment.

The present disclosure relates to new compositions, and their application as pharmaceuticals for treating disease. Methods of treating hyperkalemic periodic paralysis, hypokalemic periodic paralysis and other diseases in a human or animal subject are also provided.

Dichlorphenamide (Keveyis®, Daranide™) is a carbonic anhydrase inhibitor approved for treating primary hyperkalemic periodic paralysis, primary hypokalemic periodic paralysis, and related variants, and has been used to treat elevated intraocular pressure (TOP). Dichlorphenamide was introduced by Merck in 1950's to treat glaucoma. Dichlorphenamide is now available as immediate-release tablets for oral administration, each containing 50 mg dichlorphenamide.

The present disclosure provides a method for treating primary hyperkalemic periodic paralysis, primary hypokalemic periodic paralysis, and related variants in a subject in need thereof. The method comprises administering to the subject a therapeutically effective amount of dichlorphenamide, or a pharmaceutically acceptable salt thereof, informing the subject to notify a medical professional if the subject experiences symptoms of cognitive impairment, and adjusting the therapeutically effective amount of dichlorphenamide, or a pharmaceutically acceptable salt thereof, when the subject is experiencing symptoms of cognitive impairment.

These and other aspects of the invention will be apparent upon reference to the following detailed description. To this end, various references are set forth herein which describe in more detail certain background information, procedures, compounds, and/or compositions, and are each hereby incorporated by reference in their entirety.

### DETAILED DESCRIPTION

Dichlorphenamide is a dichlorinated benzenedisulfonamide, known chemically as 4,5-dichloro-1,3-benzenedisulfonamide. Its empirical formula is C₆H₆Cl₂N₂O₄S₂ and its structural formula is:

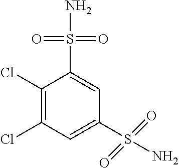 [SMILES: NS(=O)(=O)c1cc(Cl)c(Cl)c(S(N)(=O)=O)c1]

Dichlorphenamide USP is a white or practically white, crystalline compound with a molecular weight of 305.16 g/mol. It is very slightly soluble in water but soluble in dilute solutions of sodium carbonate and sodium hydroxide. Dilute alkaline solutions of dichlorphenamide are stable at room temperature. Dichlorphenamide is storage-stable for at least 36 months.

A formulation of dichlorphenamide has been previously reported in the United States Food and Drug Administration (FDA) approved drug label for Keveyis®, which is indicated for treating primary hyperkalemic periodic paralysis (“hyper” PP), primary hypokalemic periodic paralysis (“hypo” PP), and related variants, a heterogenous group of conditions for which responses may vary. The initial dose is usually 50 mg/day twice daily (bis in diem, BID), which may be adjusted at weekly intervals up to 200 mg/day.

Dichlorphenamide is a carbonic anhydrase inhibitor. The precise mechanism by which dichlorphenamide exerts its therapeutic effects in patients with periodic paralysis is unknown. It is hypothesized that dichlorphenamide modulates pH, which affects the resting membrane potential on muscle surfaces. In both hypo PP and hyper PP, skeletal muscle fibers intermittently become refractory to signals from motor neurons, leading to muscle weakness or flaccid paralysis.

When introducing elements of the present disclosure or the embodiment(s) thereof, the articles “a”, “an”, “the” and “said” are intended to mean that there are one or more of the elements. The terms “comprising”, “including” and “having” are inclusive and mean that there may be additional elements other than the listed elements.

The term “and/or” when in a list of two or more items, means that any of the listed items can be employed by itself or in combination with one or more of the listed items. For example, the expression “A and/or B” means either or both of A and B, i.e. A alone, B alone or A and B in combination. The expression “A, B and/or C” is intended to mean A alone, B alone, C alone, A and B in combination, A and C in combination, B and C in combination or A, B, and C in combination.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about” qualifies the numerical values that it modifies, denoting such a value as variable within a margin of error. When no margin of error, such as a standard deviation to a mean value given in a chart or table of data, is recited, the term “about” means that range which would encompass the recited value and the range which would be included by rounding up or down to that figure, considering significant figures.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treating a disease or disorder or on the effecting of a clinical endpoint.

The term “therapeutically acceptable” refers to those compounds (or salts, prodrugs, tautomers, zwitterionic forms, etc.) suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease. In certain embodiments, prevention of a disease may involve prevention of attacks of an intermittent nature, as well as prevention of a permanent state of muscle weakness, such as an irreversible state of impairment owing to underlying disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

As used herein, a patient is said to “tolerate” a dose of a compound if administering that dose to that patient does not result in an unacceptable adverse event or an unacceptable combination of adverse events. One of skill in the art will appreciate that tolerance is a subjective measure and that what may be tolerable to one patient may not be tolerable to a different patient. For example, one patient may not be able to tolerate headache, whereas a second patient may find headache tolerable but is not able to tolerate vomiting, whereas for a third patient, either headache alone or vomiting alone is tolerable, but the patient is not able to tolerate the combination of headache and vomiting, even if the severity of each is less than when experienced alone.

As used herein, an “adverse event” is an untoward medical occurrence associated with treatment with a substrate of a drug transporter.

As used herein, “up-titration” of a compound refers to increasing the amount of a compound to achieve a therapeutic effect that occurs before dose-limiting intolerability for the patient. Up-titration can be achieved in one or more dose increments, which may be the same or different.

The term “prodrug” refers to a compound that is made more active in vivo. Certain compounds disclosed herein may also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

While the disclosed compounds may be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound disclosed herein or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately before use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds may be a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

In addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

In certain embodiments, the subject may receive a dose of between 50 mg twice daily and to 100 mg twice daily. In certain embodiments, the dose is 50 mg once daily. In certain embodiments, the dose is 50 mg once every other day. In certain embodiments, the dose is 25 mg once daily. In certain embodiments, the dose is 25 mg once every other day.

In certain embodiments, the therapeutically effective amount of the dichlorphenamide, or a pharmaceutically acceptable salt thereof, is between 25 mg and 200 mg per day.

In certain embodiments, the therapeutically effective amount of the dichlorphenamide, or a pharmaceutically acceptable salt thereof, is 50 mg twice daily.

In certain embodiments, the dichlorphenamide, or a pharmaceutically acceptable salt thereof, is administered via a titration scheme that comprises the up-titration of the dichlorphenamide, or a pharmaceutically acceptable salt thereof, at about weekly intervals until a modified dose is administered.

In certain embodiments, the modified dose of the dichlorphenamide, or a pharmaceutically acceptable salt thereof, is 200 mg. In certain embodiments, the modified dose of the dichlorphenamide, or a pharmaceutically acceptable salt thereof, is 150 mg. In certain embodiments, the modified dose of the dichlorphenamide, or a pharmaceutically acceptable salt thereof, is 100 mg.

In certain embodiments, the dichlorphenamide, or a pharmaceutically acceptable salt thereof, is administered via a titration scheme that comprises administering a first dose of the of the dichlorphenamide, or a pharmaceutically acceptable salt thereof, for a period of one week; further increasing the dose by an amount equal to an incremental value; and determining whether the subject tolerates the further increased dose; wherein the cycle is repeated so long as the subject tolerates the further increased dose, wherein the incremental value at each cycle repetition is the same or different; and wherein if the subject does not tolerate the further increased dose, the modified dose for the subject is equal to the difference between the further increased dose and the incremental value for the last cycle repetition.

The amount of active ingredient that may be combined with the carrier materials to produce a single dosage form will vary depending upon the host treated and the mode of administration.

The compounds can be administered in various modes, e.g. orally, topically, or by injection. The precise amount of compound administered to a patient will be the responsibility of the attendant physician. In certain embodiments, the specific dose level for any patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In any case, multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few min to four weeks.

In certain embodiments, the disease is chosen from primary hyperkalemic periodic paralysis, primary hypokalemic periodic paralysis, and related variants; Aland Island eye disease atrial fibrillation, Brugada syndrome, cardiomyopathy, cerebellar syndrome, cone-rod dystrophy, cystoid macular edema of retinitis pigmentosa, Dravet syndrome, epilepsy, epileptic encephalopathy, episodic ataxia, myokymia syndrome, episodic pain syndrome, hemiplegic migraine, febrile seizures, heart block, intracranial hypertension, long QT syndrome, neuropathy, night blindness, paroxysmal exercise-induced dyskinesia, Rett syndrome, sick sinus syndrome, spinocerebellar ataxia, sudden infant death syndrome (SIDS), Timothy syndrome, ventricular fibrillation, and paroxysmal kinesigenic choreoathetosis.

In certain embodiments, the disease is chosen from primary hyperkalemic periodic paralysis, primary hypokalemic periodic paralysis, and related variants. In certain embodiments, the disease is primary hyperkalemic periodic paralysis. In certain embodiments, the disease is primary hypokalemic periodic paralysis. In certain embodiments, the disease is a related variant to primary hyperkalemic periodic paralysis. In certain embodiments, the disease is a related variant to primary hypokalemic periodic paralysis.

In certain embodiments, the disease is Aland Island eye disease.

In certain embodiments, the disease is atrial fibrillation, such as familial atrial fibrillation.

In certain embodiments, the disease is Brugada syndrome, such as type 1 or type 3.

In certain embodiments, the disease is cardiomyopathy, such as dilated cardiomyopathy.

In certain embodiments, the disease is cerebellar syndrome in phosphomannomutase 2 (PMM2) deficiency, a congenital disorder of glycosylation.

In certain embodiments, the disease is cone-rod dystrophy, such as X-linked cone-rod dystrophy.

In certain embodiments, the disease is cystoid macular edema of retinitis pigmentosa.

In certain embodiments, the disease is Dravet syndrome.

In certain embodiments, the disease is epilepsy, such as generalized epilepsy, epilepsy type two, or epilepsy with febrile seizures.

In certain embodiments, the disease is epileptic encephalopathy, early infantile epileptic encephalopathy, which is an autosomal dominant form of the disease.

In certain embodiments, the disease is episodic ataxia, such as type 1, type 2, or type 5, or myokymia syndrome

In certain embodiments, the disease is episodic pain syndrome, such as familial episodic pain syndrome.

In certain embodiments, the disease is hemiplegic migraine types, familial hemiplegic migraine types 1 and 3.

In certain embodiments, the disease is febrile seizures, such as familial febrile seizures.

In certain embodiments, the disease is heart block, such as nonprogressive heart block, and progressive heart block type IA.

In certain embodiments, the disease is intracranial hypertension, such as idiopathic intracranial hypertension.

In certain embodiments, the disease is long QT syndrome-3

In certain embodiments, the disease is neuropathy, hereditary neuropathy, sensory neuropathy, and autonomic neuropathy type VII.

In certain embodiments, the disease is night blindness, such as congenital stationary night blindness, and X-linked night blindness.

In certain embodiments, the disease is paroxysmal exercise-induced dyskinesia.

In certain embodiments, the disease is Rett syndrome.

In certain embodiments, the disease is sick sinus syndrome.

In certain embodiments, the disease is spinocerebellar ataxia, such as spinocerebellar ataxia type 6.

In certain embodiments, the disease is sudden infant death syndrome (SIDS).

In certain embodiments, the disease is Timothy syndrome.

In certain embodiments, the disease is ventricular fibrillation, such as familial ventricular fibrillation.

In certain embodiments, the disease is paroxysmal kinesigenic choreoathetosis.

In certain embodiments, the method further comprises informing the subject or a medical care worker that administration of dichlorphenamide, or a pharmaceutically acceptable salt thereof, may result in symptoms of cognitive impairment.

In certain embodiments, the symptoms of cognitive impairment comprise confusion and/or memory lapse. In certain embodiments, the symptom of cognitive impairment is confusion. In certain embodiments, the symptom of cognitive impairment is memory lapse. In certain embodiments, the symptom of cognitive impairment is a combination of confusion and memory lapse. In certain embodiments, the symptoms of cognitive impairment comprise slowed cognition, dulled cognition, foggy headedness, the inability to think clearly, and anomic aphasia. Patients have frequently reported slowed or dulled cognition (e.g., being foggy headed), and an inability to think clearly. Patients have also reported a difficulty articulating thoughts (anomic aphasia).

In certain embodiments, adjusting the therapeutically effective amount of the dichlorphenamide, or a pharmaceutically acceptable salt thereof, comprises reducing the amount of the dichlorphenamide, or a pharmaceutically acceptable salt thereof, being administered.

In certain embodiments, the method further comprises reducing the dose and/or frequency of dichlorphenamide administration, or a pharmaceutically acceptable salt thereof, administered to the subject based on the subject's ability to tolerate one or more exposure-related adverse reactions associated with the dichlorphenamide, or a pharmaceutically acceptable salt thereof.

In certain embodiments, the dose of the dichlorphenamide, or a pharmaceutically acceptable salt thereof, is decreased. In certain embodiments, the dose of the dichlorphenamide, or a pharmaceutically acceptable salt thereof, is reduced by at least 5%, by at least 10%, by at least 20%, by at least 25%, by at least 30%, by at least 40%, by at least 50%, by at least 60%, by at least 70%, by at least 75%, by at least 80%, or by at least 90%. In certain embodiments, the frequency of administration of the dichlorphenamide, or a pharmaceutically acceptable salt thereof, is decreased. For example, when the dose is not reduced, the frequency of administration might be extended from twice daily (BID) once daily (QD), or every other day (QOD), and on. In certain embodiments, the method further comprises discontinuing administration of the dichlorphenamide, or a pharmaceutically acceptable salt.

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example

A study was conducted to further characterize the time course, intensity, and outcomes of paresthesia and cognition-related adverse events during 61 weeks of dichlorphenamide treatment for primary periodic paralysis. Data were analyzed from a 9-week Phase III, randomized, double-blind, placebo-controlled phase and a 52-week open-label extension phase. The trial included two substudies: one in adults with hyperkalemic periodic paralysis and one in adults with hypokalemic periodic paralysis. The substudy data were pooled. Medical Dictionary for Regulatory Activities high-level group terms of deliria (including confusion) and mental impairment disorders were pooled for cognition-related events. Kaplan-Meier methods were used to estimate the percentages of patients with no occurrence of the adverse event over time.

Patients were randomly assigned to receive dichlorphenamide, either at their current dichlorphenamide dose if taking before the study start or 50 mg twice daily, or placebo during the double-blind phase. Dose reductions were allowed for intolerable adverse events. During the open-label phase, patients received dichlorphenamide 50 mg twice daily or continued at the same dose they were taking at the end of the 9-week double-blind phase, with dose adjustments permitted. Adverse events reported during dichlorphenamide exposure were summarized by double-blind treatment/open label treatment (placebo/dichlorphenamide or dichlorphenamide/dichlorphenamide) group.

Two of 65 patients who received double-blind treatment with placebo discontinued from the study before the extension phase; therefore, 63 patients were included in the analysis (placebo/dichlorphenamide, n=27; dichlorphenamide/dichlorphenamide, n=36) of cognition-related adverse events during dichlorphenamide exposure. Most of the 63 patients were male (61.9%), white (84.1%), and had hypokalemic periodic paralysis (68.3%). The age range was 19 to 76 years.

Overall, 16 of 63 (25.4%) patients treated with dichlorphenamide had at least one cognition-related adverse event during the 61-week study duration (Table 1). Most cognition-related adverse events were mild in intensity. Four of the 63 patients (6.3%) discontinued from the study due to a cognition-related adverse event. Six of 16 (37.5%) patients with cognition-related adverse event had the dose of dichlorphenamide reduced and the cognition-related adverse event was resolved in all six cases. The onset of cognition-related adverse events typically occurred during the first four weeks of dichlorphenamide treatment.

TABLE 1
Characterization of Cognition-Related Adverse
Events During Dichlorphenamide Treatment

| Cognition-Related Adverse Events | Patient, n (%) (n = 63) | |
|---|---|---|
| Any cognition-related adverse event | 16 | (25.4) |
| Any drug-related cognition-related adverse event* | 16 | (25.4) |
| Discontinuation due to | 4 | (6.3) |
| cognition-related adverse event | | |
| Adverse event intensity | | |
| Mild | 9 | (14.3) |
| Moderate | 5 | (7.9) |
| Severe | 2 | (3.2) |
| Resolution of last cognition-related adverse event | | |
| Resolved | 11 | (17.5) |
| Under treatment/observation | 5 | (7.9) |

*Considered by investigators to be at least possibly related to study drug.

In summary, cognition-related adverse events tended to be mild or moderate in intensity and often occurred within the first month of therapy. These adverse events uncommonly resulted in study discontinuation and were sometimes managed by reduced dichlorphenamide doses. Reduced doses frequently resolved these adverse events, suggesting a potential intervention to hasten resolution.

The various embodiments described above can be combined to provide further embodiments. All U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating primary hyperkalemic periodic paralysis, primary hypokalemic periodic paralysis, and related variants in a subject in need thereof, the method comprising:
administering to the subject an initial dose of dichlorphenamide, or a pharmaceutically acceptable salt thereof wherein the initial dose is 50 mg once daily, and
informing the subject to notify a medical professional if the subject experiences symptoms of memory lapse.

2. The method of claim 1, further comprising informing the subject or a medical care worker that administration of the dichlorphenamide, or a pharmaceutically acceptable salt thereof, may result in symptoms of memory lapse.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A method for treating primary hyperkalemic periodic paralysis, primary hypokalemic periodic paralysis, and related variants in a subject in need thereof, the method comprising:
administering to the subject an initial dose of dichlorphenamide, or a pharmaceutically acceptable salt thereof, wherein the initial dose is 50 mg twice daily,
informing the subject to notify a medical professional if the subject experiences symptoms of memory lapse, and
if the subject experiences memory lapse, reducing the dose of dichlorphenamide, or a pharmaceutically acceptable salt there, to 50 mg once daily.

17. The method of claim 1, wherein if the subject does not experience memory lapse, the dose of dichlorphenamide, or a pharmaceutically acceptable salt thereof, is increased to 50 mg twice daily.
